(19) United States
(12) Patent Application Publication
(10) Pub. No.: US 2018/0221600 A1
(43) Pub. Date: Aug. 9, 2018

(54) Metered-Dose Inhaler Counter (MDI) with Lateral Switches and Metered-Does Inhaler Including such a Counter

(71) Applicant: PRESSPART GMBH & CO. KG, Marsberg (DE)

(72) Inventors: Dana Shears, Cary, NC (US); Wayne Meng, Fogelsville, PA (US); Antonio Wilson Boyer, Macungie, PA (US)

(21) Appl. No.: 15/750,961

(22) PCT Filed: Sep. 1, 2016
(86) PCT No.: PCT/EP2016/070595

§ 371 Date: Feb. 7, 2018

Related U.S. Application Data
(60) Provisional application No. 62/214,290, filed on Sep. 4, 2015.

(51) Int. Cl. A61M 15/00; A61M 11/02; A61M 16/20

(52) U.S. Cl. CPC A61M 15/008; A61M 15/009; A61M 2205/583; A61M 16/20; A61M 2205/3327; A61M 11/02

(57) ABSTRACT

A dose counter for a metered-dose inhaler (MDI) has an actuator housing and canister with an activation valve at a valve end of the canister. The dose counter includes a circuit assembly having a substrate with at least a first and a second switch thereon. The first and second switches are sized and positioned to interact with the valve end of the canister when the canister moves from a rest position to an activation position, such that the first switch is triggered when the canister reaches a first longitudinal position and the second switch is triggered when the canister reaches a second longitudinal position that is offset from the first longitudinal position during movement of the canister from the rest position to the activation position. A counting circuit is configured to receive a signal from the first and second switches and to determine when the metered dose inhaler is activated.

### FIELD OF THE INVENTION

The present invention relates to a metered-dose inhaler (MDI) counter, to metered dose inhalers including the metered dose inhaler counter, and to related methods.

### BACKGROUND

Metered-dose inhalers (MDIs) are medication delivery devices that deliver a pharmaceutical formulation including one or more pharmaceutically active compounds (“active ingredients”) to a human or other mammalian patient.

Typically the pharmaceutical formulation is delivered by the metered-dose inhaler (MDI) as unit doses in the form of an aerosol. Each actuation of the metered-dose inhaler (MDI) delivers one unit dose. The unit dose is expelled by the metered-dose inhaler (MDI) and is taken into the body of the patient on inhalation, via the nose or mouth. The pharmaceutical formulation is delivered to or via the respiratory tract, notably to the lungs, of the patient on inhalation.

The metered-dose inhaler (MDI) includes a metering valve which is configured to ensure that each dose of the pharmaceutical formulation expelled by the metered-dose inhaler (MDI) is the same, within permitted tolerances. In particular, each dose should include the same amount of the active ingredient(s). Generally, the metering valve is configured to dispense a constant volume of the pharmaceutical formulation on each actuation of the metered-dose inhaler (MDI).

A metered-dose inhaler (MDI) dose may become less accurate after the metered-dose inhaler (MDI) has been used more than the recommended number of times. Patients typically have difficulty tracking the number of doses that they have used on a metered-dose inhaler (MDI). Although efforts have been made to provide mechanical dose counters, these dose counters may add significant cost and materials to the device and may be inaccurate. Mechanical dose counters may not be able to differentiate events when a dose is actually delivered as compared with other events, such as when a metered-dose inhaler is dropped on the ground or otherwise experiences movement that does not press the metering valve sufficiently for a dose to be delivered. Hence mechanical dose counters have not gained widespread acceptance from healthcare providers. Electro-mechanical and electronic dose counters have also been proposed but have yet to achieve a sufficiently low cost and sufficiently high reliability.

### SUMMARY OF EMBODIMENTS OF THE INVENTION

In some embodiments, a dose counter for a metered-dose inhaler has an actuator housing and canister with an activation valve at a valve end of the canister. The canister is configured to be received in the actuator housing and to move from a rest position to an activation position in which the valve is depressed against a bottom portion of the actuator housing. The dose counter includes a circuit assembly positioned on the bottom portion of the actuator housing. The circuit assembly includes a substrate with at least a first and a second switch thereon. The first and second switches are sized and positioned to interact with the valve end of the canister when the canister moves from the rest position to the activation position, such that the first switch is triggered when the canister reaches a first longitudinal position and the second switch is triggered when the canister reaches a second longitudinal position that is offset from the first longitudinal position during movement of the canister from the rest position to the activation position. The circuit assembly further includes a counting circuit that is configured to receive a signal from the first and second switches indicating at least a first time when the first switch is triggered by the canister and a second time when the second switch is triggered by the canister, and to determine when the metered dose inhaler is activated responsive to the first and second time.

In some embodiments, the first and second switches are mounted on the substrate and the first switch comprises a first switch end that extends away from the bottom wall of the actuator housing and triggers the first switch when the first switch end is depressed a first distance toward the bottom wall, and the second switch comprises a second switch end that extends away from the bottom wall of the actuator housing and triggers the second switch when the second switch end is depressed a second distance toward the bottom wall, wherein the first distance is different from the second distance.

In some embodiments, in the first longitudinal position, the first switch is activated by the canister without activating the second switch.

In some embodiments, the first switch has a height that is offset from a height of the second switch.

In some embodiments, the first switch is configured to trigger at a first depression distance and the second switch is configured to trigger at a second depression distance that is offset from the first depression distance.

In some embodiments, the counting circuit is configured to determine when the metered dose inhaler is activated responsive to the first and second times from the first and second switches such that the counting circuit increments a dose count if the first and second times have a time difference that is less than a threshold amount indicating that the canister is moving at a sufficient speed to activate the canister.

In some embodiments, the circuit assembly comprises a generally arcuate shape having an opening that receives the canister valve during operation.

In some embodiments, when the counting circuit determines when the metered dose inhaler is activated responsive to the first and second time, the counting circuit increments a counting indicia, and displays the counting indicia on the display.

In some embodiments, the dose counter further includes an accelerometer in communication with the counting circuit, and the accelerometer is configured to activate the counting circuit when the accelerometer is moved with sufficient movement to indicate shaking of the metered-dose inhaler.

In some embodiments, a metered-dose inhaler (MDI) assembly includes a metered-dose inhaler (MDI) having an actuator housing and canister with an activation valve at a valve end of the canister. The canister is configured to be received in the actuator housing and to move from a rest position to an activation position in which the valve is depressed against a bottom portion of the actuator housing. A dose counter in the actuator housing includes a circuit assembly positioned on the bottom portion of the actuator housing. The circuit assembly includes a substrate with at least a first and a second switch thereon. The first and second switches are sized and positioned to interact with the valve end of the canister when the canister moves from the rest position to the activation position, such that the first switch is triggered when the canister reaches a first longitudinal position and the second switch is triggered when the canister reaches a second longitudinal position that is offset from the first longitudinal position during movement of the canister from the rest position to the activation position. The circuit assembly further includes a counting circuit that is configured to receive a signal from the first and second switches indicating at least a first time when the first switch is triggered by the canister and a second time when the second switch is triggered by the canister, and to determine when the metered dose inhaler is activated responsive to the first and second time.

In some embodiments, the first and second switches are mounted on the substrate and the first switch comprises a first switch end that extends away from the bottom wall of the actuator housing and triggers the first switch when the first switch end is depressed a first distance toward the bottom wall, and the second switch comprises a second switch end that extends away from the bottom wall of the actuator housing and triggers the second switch when the second switch end is depressed a second distance toward the bottom wall, wherein the first distance is different from the second distance.

In some embodiments, in the first longitudinal position, the first switch is activated by the canister without activating the second switch.

In some embodiments, the first switch has a height that is offset from a height of the second switch.

In some embodiments, the first switch is configured to trigger at a first depression distance and the second switch is configured to trigger at a second depression distance that is offset from the first depression distance.

In some embodiments, the counting circuit is configured to determine when the metered dose inhaler is activated responsive to the first and second times from the first and second switches such that the counting circuit increments a dose count if the first and second times have a time difference that is less than a threshold amount indicating that the canister is moving at a sufficient speed to activate the canister.

In some embodiments, the circuit assembly comprises a generally arcuate shape having an opening that receives the canister valve during operation.

In some embodiments, when the counting circuit determines when the metered dose inhaler is activated responsive to the first and second time, the counting circuit increments a counting indicia, and displays the counting indicia on the display.

In some embodiments, the dose counter further includes an accelerometer in communication with the counting circuit, and the accelerometer is configured to activate the counting circuit when the accelerometer is moved with sufficient movement to indicate shaking of the metered-dose inhaler.

In some embodiments, the counting circuit is further configured to generate usage data regarding usage of the metered-dose inhaler (MDI), and the assembly further includes a data transmitter for receiving usage data from the counting circuit and transmitting the usage data to a remote processor. In some embodiments, the usage data comprises a number of administered doses, a date and/or time of each of the administered doses, a low and/or no dose indication and/or an indication of whether the metered-dose inhaler was shaken prior to administration of a dose.

### BRIEF DESCRIPTION OF THE DRAWINGS

The accompanying drawings, which are incorporated in and constitute a part of the specification, illustrate embodiments of the invention and, together with the description, serve to explain principles of the invention.

FIG. 1 is a perspective view of a metered-dose inhaler assembly according to some embodiments.

FIG. 2 is an exploded perspective view of the metered-dose inhaler assembly of FIG. 1.

FIG. 3 is an exploded perspective view of a dose counter and display assembly of the metered-dose inhaler assembly of FIG. 1.

FIG. 4 is a top perspective view of the dose counter of FIG. 3.

FIG. 5 is a bottom perspective view of the dose counter of FIG. 3.

FIG. 6 is a side cut-away view of the metered-dose inhaler assembly of FIG. 1.

FIG. 7 is a front cut-away view of the metered-dose inhaler assembly of FIG. 1 in a rest position.

FIG. 8 is a front cut-away view of the metered-dose inhaler assembly of FIG. 1 in a partially-activated position.

FIG. 9 is a front cut-away view of the metered-dose inhaler assembly of FIG. 1 in a fully-activated position.

FIG. 10 is a schematic diagram of the switches, counting circuit and display of the circuit assembly of the metered-dose inhaler assembly of FIG. 1.

FIG. 11 is a flowchart of operations according to some embodiments.

### DETAILED DESCRIPTION OF EMBODIMENTS OF THE INVENTION

The present invention now will be described hereinafter with reference to the accompanying drawings and examples, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

Like numbers refer to like elements throughout. In the figures, the thickness of certain lines, layers, components, elements or features may be exaggerated for clarity.

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is inverted, elements described as “under” or “beneath” other elements or features would then be oriented “over” the other elements or features. Thus, the exemplary term “under” can encompass both an orientation of “over” and “under.” The device may be otherwise oriented (rotated 90 degrees or at other orientations) and the spatially relative descriptors used herein interpreted accordingly. Similarly, the terms “upwardly,” “downwardly,” “vertical,” “horizontal” and the like are used herein for the purpose of explanation only unless specifically indicated otherwise.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present invention. The sequence of operations (or steps) is not limited to the order presented in the claims or figures unless specifically indicated otherwise.

The present invention is described below with reference to block diagrams and/or flowchart illustrations of methods, apparatus (systems) and/or computer program products according to embodiments of the invention. It is understood that each block of the block diagrams and/or flowchart illustrations, and combinations of blocks in the block diagrams and/or flowchart illustrations, can be implemented by computer program instructions. These computer program instructions may be provided to a processor of a general purpose computer, special purpose computer, and/or other programmable data processing apparatus or circuit to produce a machine, such that the instructions, which execute via the processor of the computer and/or other programmable data processing apparatus, create means for implementing the functions/acts specified in the block diagrams and/or flowchart block or blocks.

These computer program instructions may also be stored in a computer-readable memory that can direct a computer or other programmable data processing apparatus to function in a particular manner, such that the instructions stored in the computer-readable memory produce an article of manufacture including instructions which implement the function/act specified in the block diagrams and/or flowchart block or blocks.

The computer program instructions may also be loaded onto a computer or other programmable data processing apparatus to cause a series of operational steps to be performed on the computer or other programmable apparatus to produce a computer-implemented process such that the instructions which execute on the computer or other programmable apparatus provide steps for implementing the functions/acts specified in the block diagrams and/or flowchart block or blocks.

Accordingly, the present invention may be embodied in hardware and/or in software (including firmware, resident software, micro-code, etc.). Furthermore, embodiments of the present invention may take the form of a computer program product on a computer-usable or computer-readable non-transient storage medium having computer-usable or computer-readable program code embodied in the medium for use by or in connection with an instruction execution system.

The computer-usable or computer-readable medium may be, for example but not limited to, an electronic, optical, electromagnetic, or semiconductor system, apparatus, or device. More specific examples (a non-exhaustive list) of the computer-readable medium would include the following: an electrical connection having one or more wires, a portable computer diskette, a random access memory (RAM), a read-only memory (ROM), an erasable programmable read-only memory (EPROM or Flash memory), an optical fiber, and a portable compact disc read-only memory (CD-ROM).

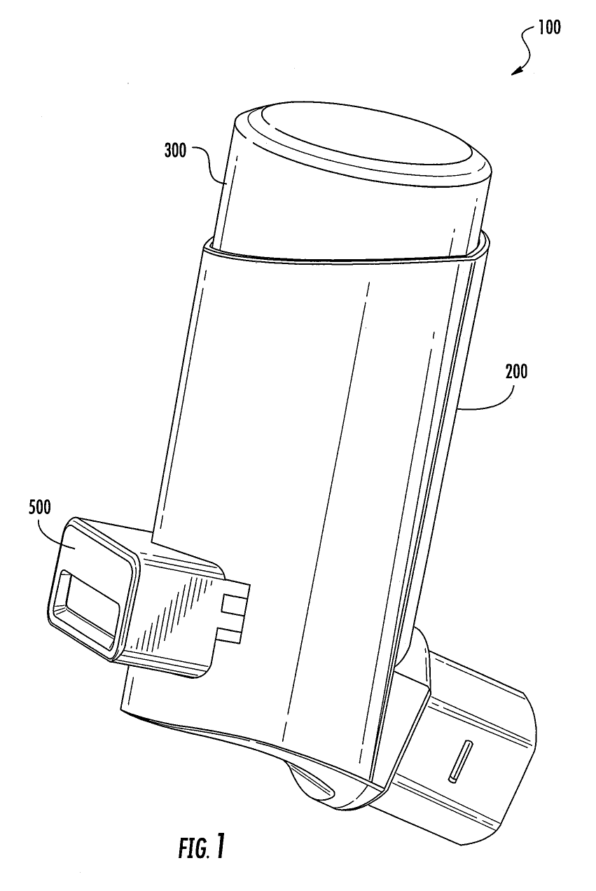

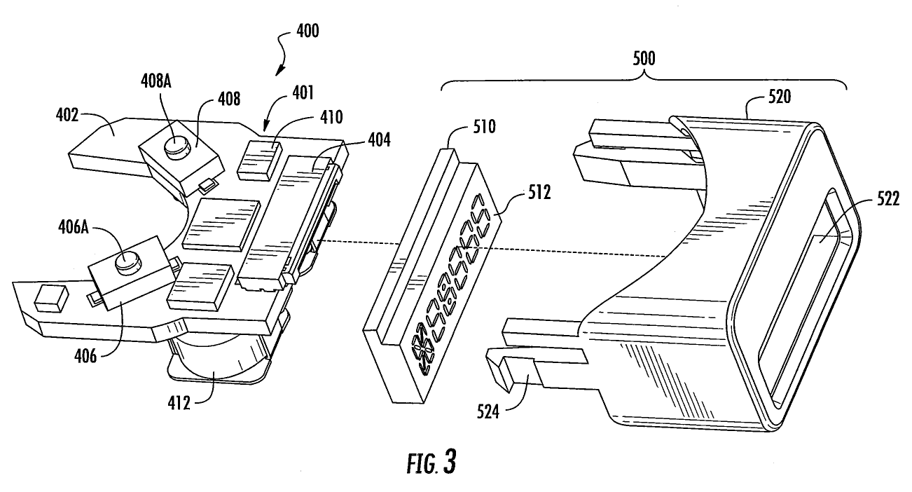

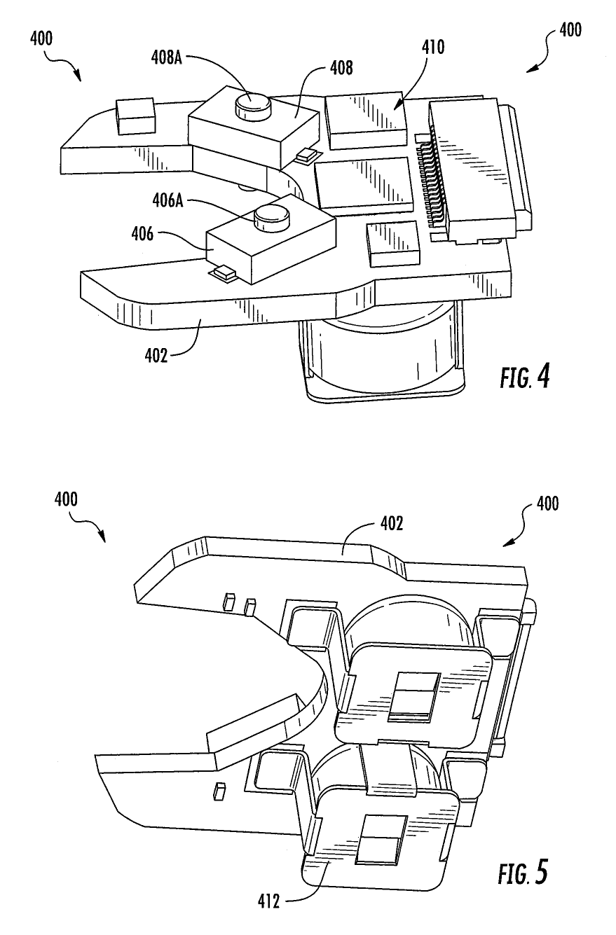

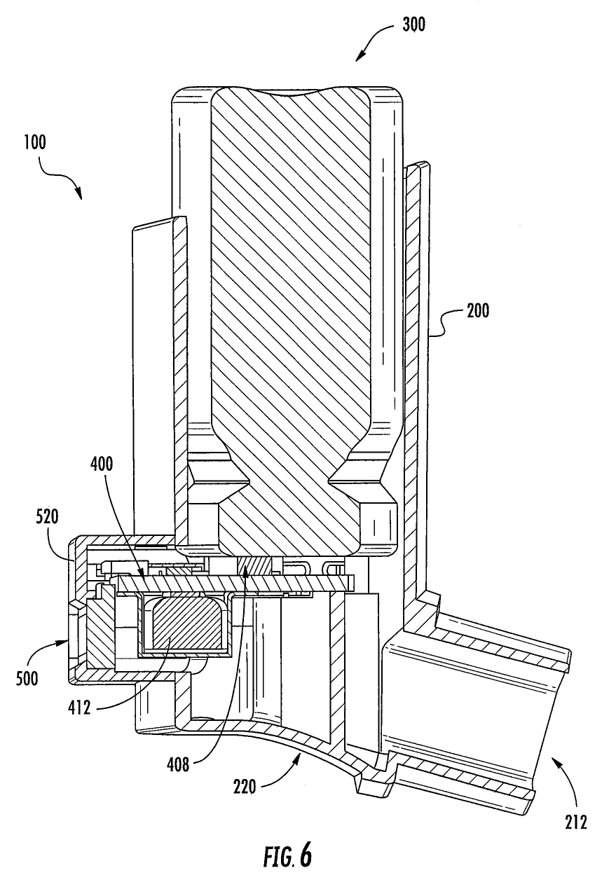

Embodiments according to the present invention will now be described with reference to FIGS. 1-11. As illustrated in FIGS. 1-9, a metered-dose inhaler (MDI) 100 includes an actuator housing 200, a canister 300, a dose counter 400 and a display assembly 500. The actuator housing 200 includes an interior cavity 210, a dispensing opening 212, a dose counter opening 214 and a bottom portion 220. The canister 300 includes an activation valve 310 on a valve end 312 thereof and another end 314 opposite the valve end 312. The dose counter 400 includes a circuit assembly 401 that has a substrate 402, a display connector 404 and at least two switches 406, 408. The dose counter 400 includes a counting circuit 410 on one side of the substrate 402 and batteries 412 on the other side of the substrate 402. The switches 406, 408 include respective switch knobs 406A, 408A that register an activation of the switch 406, 408 when depressed. The display assembly 500 includes a display 510 with a display face 512 and a display housing 520 having an opening 522 and attachment arms 524.

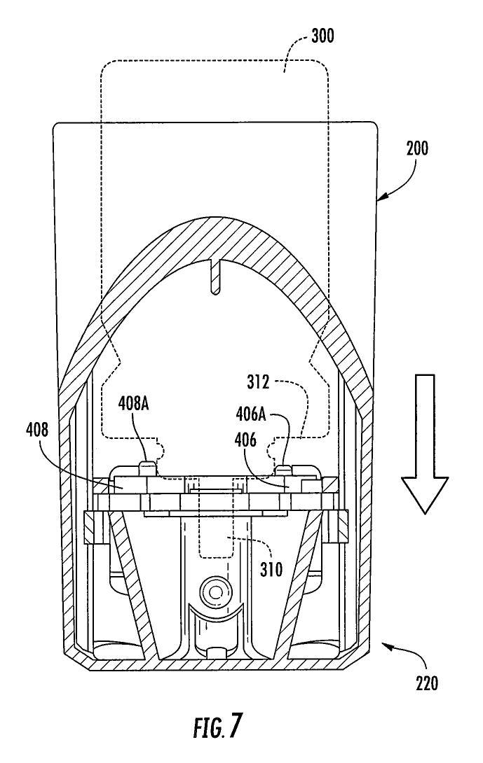

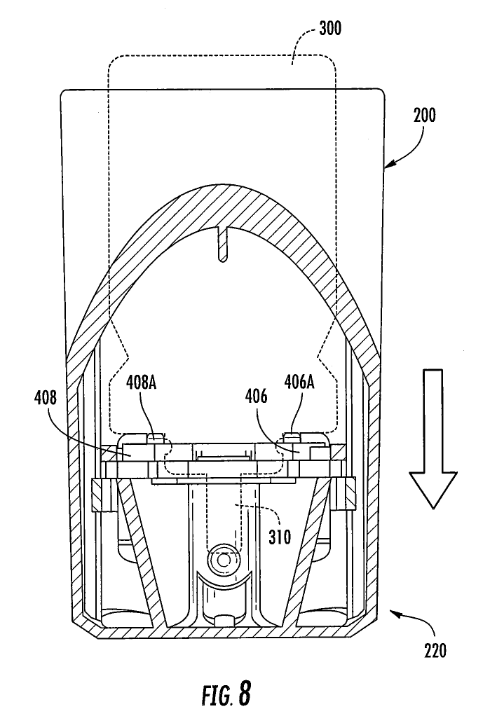

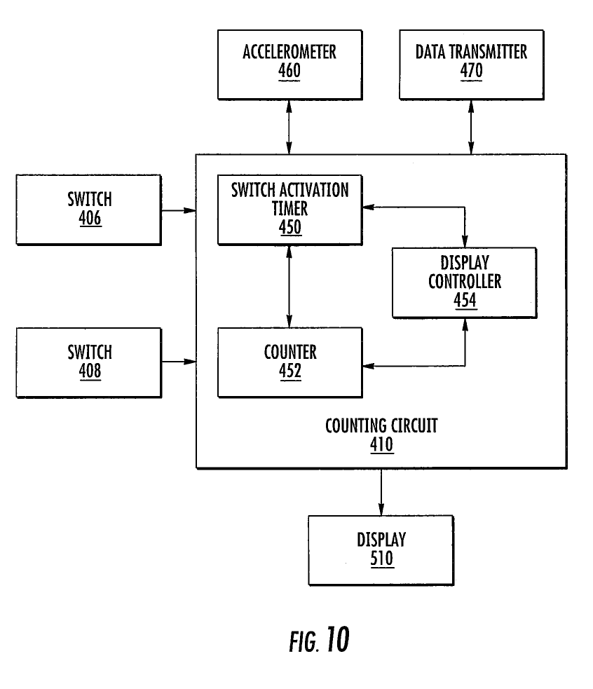

The canister 300 is received in the actuator housing cavity 210 and, as illustrated in FIGS. 7-9, the canister 300 is configured to move from a rest position (FIG. 6) to an activation position (FIG. 9) in which the valve 310 is depressed against the bottom portion 220 of the actuator housing 200 when the user presses against the end 312 of the canister 300. The circuit assembly 401 is positioned on the bottom portion 220 of the actuator housing 200 and is secured in position by the attachment arms 524 of the display housing 520.

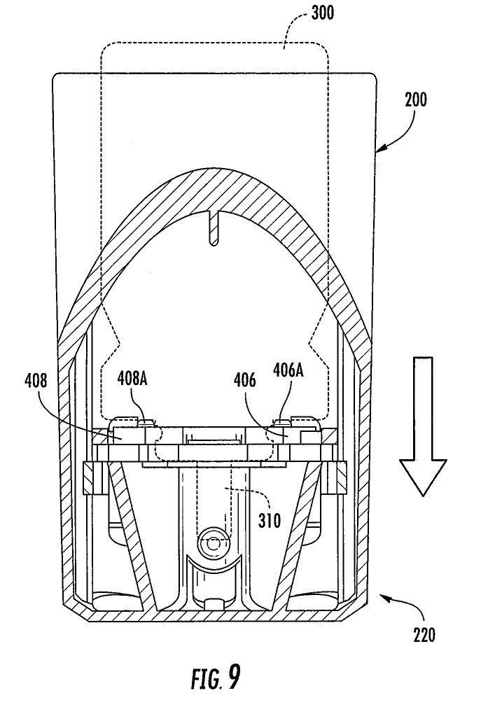

The switches 406, 408 are sized and positioned to interact with the valve end 312 of the canister 300 when the canister 300 moves from the rest position (FIG. 7) to the activation position (FIG. 9) such that one switch 408 is triggered when the canister 300 moves downward and reaches the longitudinal position shown in FIG. 8 without depressing the other switch 406. The switch 406 is triggered when the canister 200 moves further downward and reaches the longitudinal position shown in FIG. 9 such that both switch knobs 406A, 406B are depressed. The longitudinal position of the canister 200 in FIG. 8 is offset from the longitudinal position of FIG. 9 such that in FIG. 8, the switch 408 is depressed without triggering depression of the other switch 406, and in FIG. 9 both switches 406, 408 are depressed.

As illustrated, the knobs 406A, 406A of the switches 406, 408 are at different heights so that the knob 408A is higher than knob 406A. Consequently, the knob 408A is depressed by the canister valve end 312 (FIG. 8) before the knob 406A (FIG. 8) during actuation of the canister valve 210. In this configuration, the switches 406, 408 trigger at different times during the actuation of the canister valve 210 (FIGS. 7-9). The counting circuit 410 is configured to receive a signal from the switches 406, 408 indicating the triggering times of the switches 406, 408, e.g., a time corresponding to when the switch 408 is triggered by being depressed by the valve end 312 and another subsequent time when the switch 406 is triggered by being depressed by the valve end 312. The counting circuit 410 is further configured to determine when the canister valve 310 is activated based on the triggering times of the switches 406, 408. For example, in some embodiments, the counting circuit 410 registers a dose count if the triggering times of the switches 406, 408 are sufficiently close together to indicate that the canister is moving at a velocity that would generate enough force to activate the valve 310.

Although the switch knobs 406A, 408A are illustrated as being at different heights in FIGS. 7-9, it should be understood that any suitable configuration of switches that trigger actuating at different canister positions during activation may be used. For example, the switch knobs 406A, 408A and switches 406, 408 may be the same size and positioned at different heights with respect to the bottom wall or portion 220 so that the knobs 406A, 408A trigger at different depression distances when depressed toward the bottom portion 220 of the actuator housing 200. As another example, the switches 406, 408 may be configured to trigger at different depression distances even if the knobs 406A, 408A are at the same height from the bottom portion 220 of the actuator housing 200. Moreover, other types of switches may be used, such as deflection switches, optical switches, force sensors (piezoelectric force sensors) and the like.

When the counting circuit 410 determines when the metered dose inhaler is activated responsive to the signals received from the switches 406, 408 (e.g., the activation times of the switches), the counting circuit 410 increments a counting indicia, and provides instructions to the display 510 to display the counting indicia on the display 510.

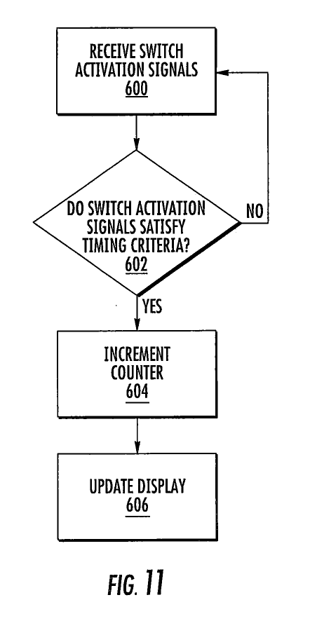

As shown in FIG. 10, the activation times of the switches 406, 408 are received as inputs to the counting circuit 410. The counting circuit 410 further includes a switch activation timer 450, a counter 452 and a display controller 454. The counting circuit 410 may also be in communication with an accelerometer 460 and a data transmitter 470.

As illustrated in FIGS. 10-11, the switch activation timer 450 receives the switch activation signals (Block 600; FIG. 11) and determines whether the switch activation signals satisfy a timing criteria (Block 602). For example, the timing criteria can be a time difference that is less than a predetermined threshold amount that indicates a successful actuation of the canister 300. The threshold amount may be an experimentally determined amount of time that indicates, for example, that the speed at which the canister 300 is depressed is sufficient to actuate the valve 310. The timing criteria may also include confirming that both switches 406, 408 have been activated. The timing criteria may be selected to reduce “false counts” or switch activation that occurs when the meter-dose inhaler (MDI) is jostled or dropped, but no dose is administered. If the timing criteria are met at Block 602, then the switch activation timer 450 increments counting indicia at the counter 452 to indicate that a dose has been dispensed (Block 604). The display controller 454 receives the counting information from the switch activation timer 450 and/or the counter 452 and updates the display 510 (Block 606). The counter 452 may increment counting indicia in either a positive or negative direction. That is, the term “increment” is meant to include both increases and decreases in counting. For example, the display 510 may display a number of doses left in the canister 300 and decrease the counter 452 when the canister 300 is depressed and the valve 308 is activated, or the display 510 may display a number of doses that have been dispensed and increase the counter 452 when the valve 310 is activated. In addition, the display controller 454 may also control the display 510 to display other information, such as an expiration date of the medication, a number of prescription refills remaining for the prescription, a time of day or a time at which the last dose was administered, and/or a message to show whether there was a sufficient dose (e.g., an error message). In particular embodiments, the display 510 is an electronic ink display, such as an electrophoretic display (E Ink Corporation, Cambridge, Mass., USA), which may reduce power consumption. However, LED displays or other suitable displays may be used.

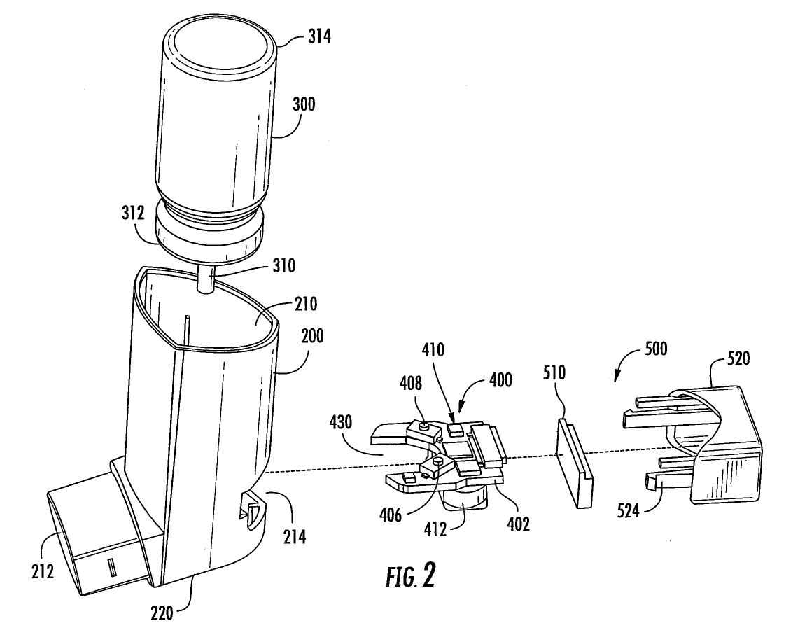

As illustrated, for example, in FIGS. 2-9, the substrate 402 or circuit board of the circuit assembly 401 may have a generally arcuate or horseshoe shape that defines an opening 430 through which the valve 310 of the canister 300 passes during activation. In this configuration, the circuit assembly 410 may be positioned within the actuator housing 200 without requiring significant additional space to reduce or eliminate the need for additional bulky counters that may inhibit ease of use and/or user acceptance of the counter 400. The display 510 and the dose counter 400 may be held in position in the actuator housing by the display housing 520 and, in particular, by the display housing arms 524, which may be positioned such that the substrate 402 rests on the arms 524. The display housing arms 524 may also engage with a corresponding engagement feature in the actuator housing 200. The display 510 is viewable by a user via the opening 522 of the housing 520.

In some embodiments, the metered-dose inhaler (MDI) assembly 100 may store and/or transmit data from the dose counter 400 using the data transmitter 470 (FIG. 10) when the device is used by the user, for example, to track patient compliance and use of the metered-dose inhaler (MDI) assembly 100. In some embodiments, the data may be transmitted by the transmitter 470 to a processor that can analyze the data, for example, for patient compliance tracking. The data can include a number of administered doses, a date and/or time of each of the administered doses, a low and/or no dose indication (e.g., based on whether the canister 300 was properly depressed) and/or an indication of whether the metered-dose inhaler was shaken prior to administration of a dose. In some embodiments, the transmitter 470 can transmit dose counts to another device remote from the dose counter 400, which can be used to analyze the data and/or transmit the data again to a network or other processor for additional analysis. The other device can be a handheld device such as a smart phone, a local computer or another medical device with additional processor capacity, such as a spirometer. The other device can either analyze the data, store the data for download, or relay the data to a computer network or other system for tracking and analysis. The transmitter 470 may be a low-energy Bluetooth connection, a radiofrequency connection, or other wireless connection and may both transmit and receive data. However, in some embodiments, a USB drive or wired connection may be used to collect and transmit the data.

As illustrated in FIG. 10, the metered-dose inhaler (MDI) assembly 100 may include an accelerometer 460 that is in communication with the dose counter counting circuit 410. The accelerometer 460 may be used to activate the dose counter 400 when the metered-dose inhaler (MDI) assembly 100 is shaken or moved by a user, for example, by activating the batteries 412 of the dose counter 400. For example, the batteries 412 may be disconnected from the circuit 410 by a switch, and the shaking of the accelerometer 460 may trigger the switch to connect the batteries 412 to the circuit 410 using a smaller power supply connected to the accelerometer 460 to conserve usage of the batteries 412. The activation of the batteries 412 by shaking helps increase the battery life of the counter 400. In some embodiments, the circuit 410 may include a timer that disconnects the batteries from the circuit 410 after a predetermined amount of time until re-activated by the accelerometer 460. The accelerometer 460 may also be used to provide data to the counting circuit 460 to indicate that the metered-dose inhaler (MDI) assembly 100 was properly shaken before use. If the dose counter 400 is moved insufficiently to properly shake the contents of the metered-dose inhaler (MDI) assembly 100 before use by the user, for example, then the signal from the accelerometer 460 may be used to activate the dose counter 400, but the data will indicate that the metered-dose inhaler (MDI) assembly 100 was not properly shaken before use. If the user properly shakes the inhaler, then the accelerometer 460 both activates the dose counter 400 (to register the dose via a force sensor or switch) and provides data indicating that the metered-dose inhaler (MDI) assembly 100 was properly shaken before use.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

1. A dose counter for a metered dose inhaler, the metered dose inhaler having an actuator housing and canister with an activation valve at a valve end of the canister, the canister being configured to be received in the actuator housing and to move from a rest position to an activation position in which the valve is depressed against a bottom portion of the actuator housing, the dose counter comprising:
a circuit assembly adapted for being positioned on the bottom portion of the actuator housing and having a substrate with at least a first and a second switch thereon, the first and second switches being sized and positioned to interact with the valve end of the canister when the canister moves from the rest position to the activation position, such that the first switch is triggered when the canister reaches a first longitudinal position and the second switch is triggered when the canister reaches a second longitudinal position that is offset from the first longitudinal position during movement of the canister from the rest position to the activation position; and
the circuit assembly further comprises a counting circuit that is configured to receive a signal from the first and second switches indicating at least a first time when the first switch is triggered by the canister and a second time when the second switch is triggered by the canister, and to determine when the metered dose inhaler is activated responsive to the first and second times from the first and second switches such that the counting circuit increments a dose count if the first and second times have a time difference that is less than a threshold amount indicating that the canister is moving at a sufficient speed to activate the canister.

2. The dose counter of claim 1, wherein the first and second switches are mounted on the substrate and the first switch comprises a first switch end that extends away from the bottom wall of the actuator housing and triggers the first switch when the first switch end is depressed a first distance toward the bottom wall, and the second switch comprises a second switch end that extends away from the bottom wall of the actuator housing and triggers the second switch when the second switch end is depressed a second distance toward the bottom wall, wherein the first distance is different from the second distance.

3. The dose counter of claim 1, wherein in the first longitudinal position, the first switch is activated by the canister without activating the second switch.

4. The dose counter of claim 1, wherein the first switch has a height that is offset from a height of the second switch.

5. The dose counter of claim 1, wherein the first switch is configured to trigger at a first depression distance and the second switch is configured to trigger at a second depression distance that is offset from the first depression distance.

6. The dose counter of claim 1, wherein the circuit assembly comprises a generally arcuate shape having an opening that receives the canister valve during operation.

7. The dose counter of claim 1, wherein when the counting circuit determines when the metered dose inhaler is activated responsive to the first and second time, the counting circuit increments a counting indicia, and displays the counting indicia on the display.

8. The dose counter of claim 1, further comprising an accelerometer in communication with the counting circuit, wherein the accelerometer is configured to activate the counting circuit when the accelerometer is moved with sufficient movement to indicate shaking of the metered-dose inhaler.

9. A metered-dose inhaler assembly comprising:
a metered dose inhaler having an actuator housing and canister with an activation valve at a valve end of the canister, the canister being configured to be received in the actuator housing and to move from a rest position to an activation position in which the valve is depressed against a bottom portion of the actuator housing;
a dose counter in the actuator housing, the dose counter comprising:
a circuit assembly positioned on the bottom portion of the actuator housing and having a substrate with at least a first and a second switch thereon, the first and second switches being sized and positioned to interact with the Valve end of the canister when the canister moves from the rest position to the activation position, such that the first switch is triggered when the canister reaches a first longitudinal position and the second switch is triggered when the canister reaches a second longitudinal position that is offset from the first longitudinal position during movement of the canister from the rest position to the activation position; and
the circuit assembly further comprises a counting circuit that is configured to receive a signal from the first and second switches indicating at least a first time when the first switch is triggered by the canister and a second time when the second switch is triggered by the canister, and to determine when the metered dose inhaler is activated responsive to the first and second times from the first and second switches such that the counting circuit increments a dose count if the first and second times have a time difference that is less than a threshold amount indicating that the canister is moving at a sufficient speed to activate the canister.

10. The metered-dose inhaler assembly of claim 9, wherein the first and second switches are mounted on the substrate and the first switch comprises a first switch end that extends away from the bottom wall of the actuator housing and triggers the first switch when the first switch end is depressed a first distance toward the bottom wall, and the second switch comprises a second switch end that extends away from the bottom wall of the actuator housing and triggers the second switch when the second switch end is depressed a second distance toward the bottom wall, wherein the first distance is different from the second distance.

11. The metered-dose inhaler assembly of claim 9, wherein in the first longitudinal position, the first switch is activated by the canister without activating the second switch.

12. The metered-dose inhaler assembly of claim 9, wherein the first switch has a height that is offset from a height of the second switch.

13. The metered-dose inhaler assembly of claim 9, wherein the first switch is configured to trigger at a first depression distance and the second switch is configured to trigger at a second depression distance that is offset from the first depression distance.

14. The metered-dose inhaler assembly of claim 9, wherein the circuit assembly comprises a generally arcuate shape having an opening that receives the canister valve during operation.

15. The metered-dose inhaler assembly of claim 9, wherein when the counting circuit determines when the metered dose inhaler is activated responsive to the first and second time, the counting circuit increments a counting indicia, and displays the counting indicia on the display.

16. The metered-dose inhaler assembly of claim 9, further comprising an accelerometer in communication with the counting circuit, wherein the accelerometer is configured to activate the counting circuit when the accelerometer is moved with sufficient movement to indicate shaking of the metered-dose inhaler.

17. The metered-dose inhaler assembly of claim 9, wherein the counting circuit is further configured to generate usage data regarding usage of the metered-dose inhaler, the assembly further comprising a data transmitter for receiving usage data from the counting circuit and transmitting the usage data to a remote processor.

18. The metered-dose inhaler assembly of claim 9, wherein the usage data comprises a number of administered doses, a date and/or time of each of the administered doses, a low and/or no dose indication and/or an indication of whether the metered-dose inhaler was shaken prior to administration of a dose.
